(19) United States
(12) Patent Application Publication
(10) Pub. No.: US 2011/0124579 A1
(43) Pub. Date: May 26, 2011

(54) SIALIC ACID TO SUPPORT SALIVATION

(75) Inventors: Norbert Sprenger, Savigny (CH); Karine Vidal, Lausanne (CH); Christine Cherbut, Pully (CH); Alfred Jann, Publier (FR)

(73) Assignee: NESTEC S.A., Vevey (CH)

(21) Appl. No.: 12/991,585

(22) PCT Filed: May 6, 2009
(86) PCT No.: PCT/EP2009/055461

§ 371 Date: Jan. 21, 2011

(30) Foreign Application Priority Data
May 8, 2008 (EP) 08155853.8

(51) Int. Cl. A61K 38/14; A61K 31/7008; A61K 31/715; A61P 1/00

(52) U.S. Cl. 514/21.1; 514/62; 514/54

(57) ABSTRACT

The present invention generally relates to the field of disorders related to an impaired salivation and compositions that can be used to treat or prevent such disorders. One embodiment of the present invention relates to a composition comprising sialic acid to treat and/or prevent disorders linked to an impaired salivation.

The present invention generally relates to the field of disorders related to an impaired salivation and compositions that can be used to treat or prevent such disorders. One embodiment of the present invention relates to a composition comprising sialic acid to treat and/or prevent disorders linked to an impaired salivation.

Many persons live to old age in good health. But many functional and structural changes occur in the body during aging, and not every person can cope with the changes easily. Reasons for the inter-individual differences are manifold and include genotype, nutrition and behaviour.

Aging is associated with functional alterations of neurons and a progressive neuronal loss of the central and peripheral nervous system.

A typical condition frequently associated with age is a dry-mouth-feeling linked to deficits in salivation.

Such a dry mouth feeling may be associated with difficulties in swallowing, which will further reduce a persons willingness to consume normal food products in sufficient amounts to sustain a good health.

There is hence a need in the art for a composition that can be used to treat or prevent problems associated with an impaired salivation.

The present inventors have addressed this need.

Consequently, it was the object of the present invention to provide the art with a composition that is available to everybody that can be administered without the risk of unwanted side effects that is inexpensive and that can be used to improve salivation, particularly in elderly people.

This object was achieved by the subject matter of the independent claims. The dependant claims further develop the present invention.

Sialic acids (SiAc) are a family of charged nine carbon monosaccharides derived from neuraminic acid (NeuAc). NeuAc is the only sialic acid normally formed in humans. In other vertebrates, for example N-glycolylneuraminic acids (NeuGc), are also present.

Today, sialic acids are frequently used in the field of infant nutrition. For example, a possible involvement of SiAc in the cognitive development of infants was summarized by Wang (Wang, B. and Brand-Miller, J. (2003) Eur. J. Clin. Nutr. November; 57(11):1351-69). Briefly, studies comparing breast-fed and formula-fed infants demonstrate that a higher NeuAc content of breast milk compared to a normal infant formula correlates with an increased NeuAc content of infants saliva and brain. However, behavioural effects of NeuAc supplementation in humans are not available. Nevertheless it is speculated that supplementation of cows milk with NeuAc would provide the cows milk with human milk attributes, which might have an impact on brain development of children.

Natural sources rich in SiAc, for example NeuAc, are, e.g., human milk, elephant milk, Indian buffalo milk, meat, eggs and fish.

The present inventors have administrated sialic acid to aged rats. Aged animals showed less salivation as compared to young animals. Surprisingly, sialic acid feeding lead to an increased stimulated salivation equal to the salivation found in young animals.

Consequently, one embodiment of the present invention is a composition comprising sialic acid for treating and/or preventing disorders linked to an impaired salivation.

The disorder linked to an impaired salivation may be selected from the group consisting of dysphagia, xerostomia and combinations thereof.

Dysphagia is a swallowing disorder characterized by difficulty in oral preparation for the swallow, or in moving material from the mouth to the stomach. This also includes problems in positioning food in the mouth. Dysphagia is due to problems in nerve or muscle control. It is common, for example, after a stroke and head/neck cancer treatment. Dysphagia compromises nutrition and hydration and may lead to aspiration pneumonia and dehydration.

Xerostomia is the medical term for dry mouth feeling due to reduced salivation function. Chronic dry mouth can be uncomfortable and lead to serious health problems. A dry mouth can cause difficulties in tasting, chewing, swallowing, and speaking. If it goes untreated, severe dry mouth can also lead to increased levels of tooth decay and infections of the mouth such as of Candida.

A preferred form of sialic acid is N-acetylneuraminic acid.

N-Acetylneuraminic acid has the following synonyms and abbreviations: o-Sialic acid; 5-Acetamido-3,5-dideoxy-D-glycero-D-galacto-2-nonulosonic acid; 5-Acetamido-3,5-dideoxy-D-glycero-D-galactonulosonic acid; Aceneuramic acid; N-acetyl-neuraminate; N-Acetylneuraminic acid; NANA, and Neu5Ac.

It may be preferred if the ingredient and/or the composition is enriched with sialic acid.

One embodiment of the present invention is a composition containing a protein fraction comprising N-acetylneuraminic acid bound to a threonine rich peptide/protein backbone for treating or preventing disorders linked to an impaired salivation.

Threonine rich means that the threonine content is higher than the average threonine abundance in the human protein mass. For example, the threonine content may be increased by at least 10% compared to the average threonine abundance in the human protein mass.

Thus threonine may account for at least 6.3 mol-% of the amino acids in the protein fraction.

For example, threonine may be present in an amount of between about 8 and 22% of the total number of amino acids.

The protein fraction may further comprise about 7 to 25% by mass N-acetylneuraminic acid.

N-acetylneuraminic acid may be provided in a glycan bound form. For example N-acetylneuraminic acid may be provided in a form bound to glycoproteins and/or proteoglycans.

According to a particular preferred embodiment of the present invention, the protein fraction comprises N-acetylneuraminic acid (NeuAc) that is characterized by a threonine rich peptide/protein backbone (between 8 and 22% of total number of amino acids) and a NeuAc content of 7 to 25% by mass.

N-acetylneuraminic acid may be provided in the form of an oligosaccharide ingredient, for example, which comprises glycosylated amino acids and peptides of the general formula RₙSacₘ where R is an amino acid residue, Sac is a monosaccharide selected from the group comprising N-acetyl-neuraminic acid, N-acetyl galactosamine and galactose, n has a value between 1 and 10 with the proviso that if n has the value 1 R is a threonine residue or a serine residue and if n has a value between 2 and 10 the peptide contains at least one threonine or serine residue, m has a value between 2 and 4 and at least 15 mol % of the ingredient is N-acetyl-neuraminic acid.

Preferably n has a value between 1 and 3 and m has a value of 3 or 4.

The ingredient contains at least 15 mol % sialic acid as part of a saccharide chain linked to the hydroxyl group of threonine or serine. The sialic acid may form part of the chain or may itself be a substituent of a monosaccharide unit in the chain.

Preferably, the oligosaccharide ingredient contains the following monosaccharides:—

| Compound | mol % |
|---|---|
| N-acetyl galactosamine | 20-25 |
| (GalNAc) | |
| galactose (Gal) | 20-25 |
| N-acetyl-neuraminic acid | 40-17.5 |
| (NeuAc) | |

The oligosaccharide ingredient may contain from 20 to 25 mol % of a mixture of serine and threonine.

The oligosaccharide ingredient may contain the following glycosylated amino acids or peptides:—

NeuAc-α-2,3-Gal-β-1,3-(NeuAc-α-2,6-)-GalNAc-Rₙ

NeuAc-α-2,3-Gal-β-1,3-GalNAc-Rₙ

Gal-β-1,3-(NeuAc-α-2,6-)-GalNAc-Rₙ

Gal-β-1,3-GalNAc-Rₙ

The oligosaccharide ingredient of the invention may be produced by the hydrolysis of CGMP using an exoprotease and an endoprotease either together or sequentially to obtain a mixture of free amino acids and peptides with a chain length between 2 and 10 and subjecting the hydrolysed mixture to nanofiltration so as to retain the fraction having a molecular weight between 1000 and 2000 Daltons.

CGMP itself is a by-product of cheese-making in which whole milk is treated with the enzyme rennin to precipitate the casein. In this process, CGMP is cleaved from κ casein and remains in solution with the whey proteins. This product is known as sweet whey. The CGMP may be separated from the whey proteins by any process known in the art. A suitable process is described in European Patent No. 986312.

Without being bound by theory the inventors suppose that a protein fraction comprising N-acetylneuraminic acid bound to a threonine rich peptide/protein backbone, for example glycan bound N-acetylneuraminic acid, is advantageous over free N-acetylneuraminic acid because of the following reasoning. Free N-acetylneuraminic acid leads to a very fast ‘acute’ uptake and systemic increase in N-acetylneuraminic acid triggering a fast reestablishment of circulating N-acetylneuraminic acid levels by higher excretion into urine. A protein fraction comprising N-acetylneuraminic acid bound to a threonine rich peptide/protein backbone avoids this. For example, glycan bound N-acetylneuraminic acid reaches all gut segments leading to a slow ‘chronic’ N-acetylneuraminic acid uptake all along the gut including lower small intestine and colon.

The composition may be administered to the elderly.

A subject is considered as “elderly” if it has surpassed the first half of its average expected lifespan in its country of origin, preferably, if it has surpassed the first two thirds of the average expected lifespan in its country of origin, more preferably if it has surpassed the first three quarters of the average expected lifespan in its country of origin, most preferred if it has surpassed the first four fifths of the average expected lifespan in its country of origin.

The composition may be administered to humans or animals, in particular pets, companion animals and/or livestock.

The composition of the present invention may be a nutritional composition, a nutraceutical, a drink, a food additive or a medicament. A food additive or a medicament may be in the form of tablets, capsules, pastilles or a liquid for example. Food additives or medicaments are preferably provided as sustained release formulations, allowing a constant SiAc supply for prolonged times.

The composition is preferably selected from the group consisting of milk powder based products; instant drinks; ready-to-drink formulations; nutritional powders; nutritional liquids; milk-based products, in particular yoghurts or ice cream; cereal products; beverages; water; coffee; cappuccino; malt drinks; chocolate flavoured drinks; culinary products; soups; topical creams; suppositories; tablets; syrups; and formulations for transdermal applications.

Milk may be any milk obtainable from animal or plant sources and is preferably cows milk, human milk, sheep milk, goat milk, horse milk, camel milk, rice milk or soy milk.

Instead of milk, also milk derived protein fractions or colostrum may be used.

The composition may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials. It may also contain conventional pharmaceutical additives and adjuvants, excipients and diluents, including, but not limited to, water, gelatine of any origin, vegetable gums, ligninsulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like. Further, it may contain an organic or inorganic carrier material suitable for oral or enteral administration as well as vitamins, minerals trace elements and other micronutrients in accordance with the recommendations of Government bodies such as the USRDA.

For example, the composition may contain per daily dose one or more of the following micronutrients in the ranges given:— 300 to 500 mg calcium, 50 to 100 mg magnesium, 150 to 250 mg phosphorus, 5 to 20 mg iron, 1 to 7 mg zinc, 0.1 to 0.3 mg copper, 50 to 200 μg iodine, 5 to 15 μg selenium, 1000 to 3000 μg beta carotene, 10 to 80 mg Vitamin C, 1 to 2 mg Vitamin B1, 0.5 to 1.5 mg Vitamin B6, 0.5 to 2 mg Vitamin B2, 5 to 18 mg niacin, 0.5 to 2.0 μg Vitamin B12, 100 to 800 μg folic acid, 30 to 70 μg biotin, 1 to 5 μg Vitamin D, 3 to 10 μg Vitamin E.

The composition of the present invention may contain a protein source, a carbohydrate source and/or a lipid source.

Any suitable dietary protein may be used, for example animal proteins (such as milk proteins, meat proteins and egg proteins); vegetable proteins (such as soy protein, wheat protein, rice protein, and pea protein); mixtures of free amino acids; or combinations thereof. Milk proteins such as casein and whey, and soy proteins are particularly preferred. Very positive results for the purpose of the present invention were achieved when the protein fraction comprised threonine in an amount of between about 8 and 22% of the total number of amino acids of the protein fraction.

If the composition includes a fat source, the fat source more preferably provides 5% to 40% of the energy of the formula; for example 20% to 30% of the energy. DHA may be added. A suitable fat profile may be obtained using a blend of canola oil, corn oil and high-oleic acid sunflower oil.

A source of carbohydrates may more preferably provide between 40% to 80% of the energy of the composition. Any suitable carbohydrate may be used, for example sucrose, lactose, glucose, fructose, corn syrup solids, maltodextrins, and mixtures thereof.

According to a particular preferred embodiment of the present invention, the protein fraction comprises N-acetylneuraminic acid (NeuAc) and is characterized by a threonine rich peptide/protein backbone (between 8 and 22% of total number of amino acids) and a NeuAc content of 7 to 25% by mass.

The composition may also comprise a probiotic micro-organism and/or a prebiotic such as fructooligosaccharides, galactosyloligosaccharides, pectins and/or hydrolysates thereof.

Prebiotics are in particular preferred if the composition comprises probiotics, since the presence of probiotics and prebiotics produces a synergistic effect.

“Probiotic” means microbial cell preparations or components of microbial cells with a beneficial effect on the health or well-being of the host. (Salminen S, Ouwehand A. Benno Y. et al “Probiotics: how should they be defined” Trends Food Sci. Technol. 1999:10 107-10).

All probiotic micro-organisms may be used in accordance with the present invention.

Preferably, they are selected from the group consisting of Bifidobacterium, Lactobacillus, Streptococcus and Saccharomyces or mixtures thereof, in particular selected from the group consisting of Bifidobacterium longum, Bifidobacterium lactis, Lactobacillus acidophilus, Lactobacillus rhamnosus, Lactobacillus paracasei, Lactobacillus johnsonii, Lactobacillus plantarum, Lactobacillus salivarius, Enterococcus faecium, Saccharomyces boulardii and Lactobacillus reuteri or mixtures thereof, preferably selected from the group consisting of Lactobacillus johnsonii (NCC533; CNCM I-1225), Bifidobacterium longum (NCC490; CNCM I-2170), Bifidobacterium longum (NCC2705; CNCM I-2618), Bifidobacterium lactis (2818; CNCM I-3446), Lactobacillus paracasei (NCC2461; CNCM I-2116), Lactobacillus rhamnosus GG (ATCC53103), Lactobacillus rhamnosus (NCC4007; CGMCC 1.3724), Enterococcus faecium SF 68 (NCIMB10415), and mixtures thereof.

“Prebiotic” means food substances that promote the growth of probiotics in the intestines. They are not broken down in the stomach and/or upper intestine or absorbed in the GI tract of the person ingesting them, but they are fermented by the gastrointestinal microflora and/or by probiotics. Prebiotics are for example defined by Glenn R. Gibson and Marcel B. Roberfroid, Dietary Modulation of the Human Colonic Microbiota: Introducing the Concept of Prebiotics, J. Nutr. 1995 125: 1401-1412.

The prebiotics that may be used in accordance with the present inventions are not particularly limited and include all food substances that promote the growth of probiotics in the intestines. Preferably, they may be selected from the group consisting of oligosaccharides, optionally containing fructose, galactose, mannose; dietary fibers, in particular soluble fibers, soy fibers; inulin; or mixtures thereof. Preferred prebiotics are fructo-oligosaccharides, galacto-oligosaccharides, isomalto-oligosaccharides, xylo-oligosaccharides, oligosaccharides of soy, glycosylsucrose, lactosucrose, lactulose, palatinose-oligosaccharides, malto-oligosaccharides, gums and/or hydrolysates thereof, pectins and/or hydrolysates thereof.

The effect of sialic acid in the composition of the present invention was found to be essentially dose dependent. Small amounts will produce smaller effects and large amounts may be to large so that the body cannot utilize all SiAc provided. The exact amount of SiAc to be provided will depend on the subject to be treated and on its condition, for example.

While in general every amount of SiAc will produce a beneficial effect it was found to be in particularly preferred if the sialic acid is present in the composition in an amount of 1 mg-250 mg/g dry mass of the composition.

The sialic acid may be administered in a daily amount of 1 mg-2 g/kg body weight, preferably 0,025 g to 0.8 g/kg body weight of the subject to be treated.

Those skilled in the art will understand that they can freely combine all features of the present invention described herein, without departing from the scope of the invention as disclosed. In particular, features described for the uses of the present invention may be applied to the composition of the present invention and vice versa.

Further advantages and features of the present invention are apparent from the following Examples and Figures.

Figures:

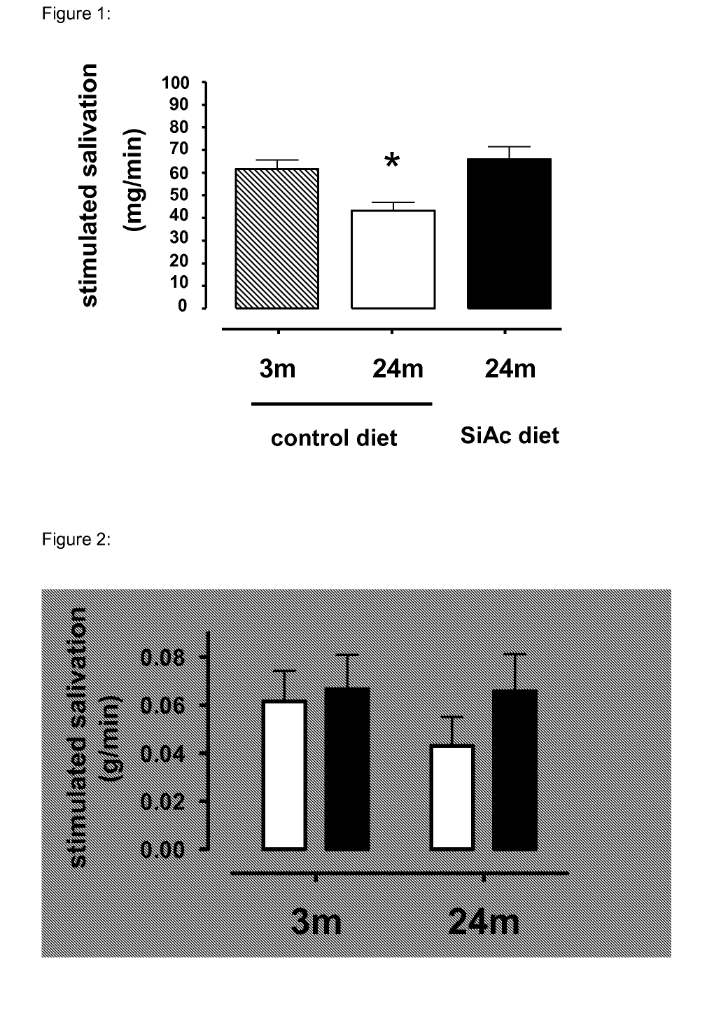

FIG. 1 demonstrates that aged rats show less cholinergic neuronal activity as measured by stimulated saliva production per time when compared to young adult rats, but show a significantly increased neuronal activity upon feeding a sialic acid rich diet. Mean values and standard error of the mean are presented. N=9-10; * indicates significant difference at a p=0.0035 comparing 3 and 24 months on control diet and at a p=0.0024 comparing 24 months control and Sia diet by t-test.

FIG. 2 shows again the Pilocarpine stimulated salivation in young adult (3 m) and old age (24 m) rats fed a control diet (open bars) or a NeuAc enriched diet (filled bars) for 3 weeks. Means and SD are shown (N=8-10). 2-way ANOVA showed significant age (p=0.0364) and treatment (p=0.0037) effects and an almost significant interaction (p=0.0550) effect. Note, that NeuAc feeding increased stimulated salivation in aged rats much more efficiently than in young adult rats.

### EXAMPLES

Young adult (3 months) and aged rats (24 months) were fed a semisynthetic diet containing sialic acid at a concentration of 0.15 g/100 g diet (control) or a semi-synthetic diet additionally supplemented with sialic acid to a final concentration of 0.9 g/100 g diet (Sia).

After 3 weeks on the experimental diet neuronal activity of cholinergic neurons was assessed. To this end rats were injected pilocarpine (IP, 1.5 mg/kg body weight for 24 months rats and 2 mg/kg body weight for 3 months rats). Pilocarpine is a muscarinic agonist acting on cholinergic neurons, which leads to salivation. Thus stimulated saliva collection was timed with a chronometer. After about 7 minutes collection was stopped.

As seen in FIGS. 1 and 2, aged animals showed less stimulated salivation as compared to young animals. Upon sialic acid feeding the aged animals reached similar stimulated salivation values as the young animals. This indicates that sialic acid feeding helped functional recovery of cholinergic neuron function that decreased with age.

The observations made here show that a proper salivation was re-established in aged animals by providing sialic acid via a nutritional approach.

1. A method for treating disorders linked to an impaired salivation comprising the steps of administering a therapeutically-effective amount of a composition comprising sialic acid to an individual having same.

2. Method in accordance with claim 1, wherein the sialic acid is N-acetylneuraminic acid.

3. Method in accordance with claim 1, wherein the sialic acid is bound to a threonine rich peptide/protein backbone.

4. Method in accordance with claim 1 comprising a protein fraction that comprises about 7 to 25% by weight sialic acid and threonine in an amount of between about 8 and 22% of the total number of amino acids.

5. Method in accordance with claim 1, wherein the sialic acid is an oligosaccharide ingredient comprising glycosylated amino acids and peptides of the general formula RₙSacₘ where R is an amino acid residue, Sac is a monosaccharide selected from the group consisting of N-acetyl-neuraminic acid, N-acetyl galactosamine and galactose, n has a value between 1 and 10 with the proviso that if n has the value 1, R is a threonine residue or a serine residue and if n has a value between 2 and 10 the peptide contains at least one threonine or serine residue, m has a value between 2 and 4 and at least 15 mol % of the ingredient is N-acetyl-neuraminic acid.

6. Method in accordance with claim 1, wherein the disorder linked to an impaired salivation is selected from the group consisting of dysphagia, xerostomia and combinations thereof.

7. Method in accordance with claim 1, wherein the composition is in a form selected from the group consisting of a pharmaceutical composition, a food product, a food additive and a nutraceutical.

8. Method in accordance with claim 1, wherein the composition further comprises an ingredient selected from the group consisting of a neuron protective agent, a probiotic and a prebiotic.

9. Method in accordance with claim 1, wherein the sialic acid is present in the composition in an amount of 1 mg to 250 mg/g dry weight of the composition.

10. Method in accordance with claim 1, wherein the sialic acid is to be administered in a daily amount of 1 mg to 2 g/kg body weight to the subject.

11. Method in accordance with claim 1, wherein the composition is selected from the group consisting of milk powder based products; instant drinks; ready-to-drink formulations; nutritional powders; nutritional liquids, milk-based products, in particular yoghurts or ice cream; cereal products; biscuits; cereal bars; beverages; water; coffee; cappuccino; tea; fruit juices; malt drinks; chocolate flavoured drinks; culinary products; soups; confectionary products; chocolates; topical creams, suppositories, tablets, syrups, and formulations for transdermal applications.

12. A method for preventing disorders linked to an impaired salivation comprising the step of administering a therapeutically-effective amount of a composition comprising sialic acid to an individual at risk of same.

13. Method in accordance with claim 12, wherein the sialic acid is N-acetylneuraminic acid.

14. Method in accordance with claim 12, wherein the sialic acid is bound to a threonine rich peptide/protein backbone.

15. Method in accordance with claim 12 comprising a protein fraction that comprises about 7 to 25% by weight sialic acid and threonine in an amount of between about 8 and 22% of the total number of amino acids.

16. Method in accordance with claim 12, wherein the sialic acid is an oligosaccharide ingredient comprising glycosylated amino acids and peptides of the general formula RₙSacₘ where R is an amino acid residue, Sac is a monosaccharide selected from the group consisting of N-acetyl-neuraminic acid, N-acetyl galactosamine and galactose, n has a value between 1 and 10 with the proviso that if n has the value 1, R is a threonine residue or a serine residue and if n has a value between 2 and 10 the peptide contains at least one threonine or serine residue, m has a value between 2 and 4 and at least 15 mol % of the ingredient is N-acetyl-neuraminic acid.

17. Method in accordance with claim 12, wherein the disorder linked to an impaired salivation is selected from the group consisting of dysphagia, xerostomia and combinations thereof.

18. Method in accordance with claim 12, wherein the composition is in a form selected from the group consisting of a pharmaceutical composition, a food product, a food additive and a nutraceutical.

19. Method in accordance with claim 12, wherein the composition further comprises an ingredient selected from the group consisting of a neuron protective agent, a probiotic and a prebiotic.

20. Method in accordance with claim 12, wherein the sialic acid is present in the composition in an amount of 1 mg to 250 mg/g dry weight of the composition.

21. Method in accordance with claim 12, wherein the sialic acid is to be administered in a daily amount of 1 mg to 2 g/kg body weight to the subject.

22. Method in accordance with claim 12, wherein the composition is selected from the group consisting of milk powder based products; instant drinks; ready-to-drink formulations; nutritional powders; nutritional liquids, milk-based products, in particular yoghurts or ice cream; cereal products; biscuits; cereal bars; beverages; water; coffee; cappuccino; tea; fruit juices; malt drinks; chocolate flavoured drinks; culinary products; soups; confectionary products; chocolates; topical creams, suppositories, tablets, syrups, and formulations for transdermal applications.
